(19) United States
(12) Patent Application Publication
(10) Pub. No.: US 2022/0265568 A1
(43) Pub. Date: Aug. 25, 2022

(54) PROCESS FOR PREPARING PRESSURE SENSITIVE ADHESIVE COMPOUNDS FOR USE IN A TRANSDERMAL THERAPEUTIC SYSTEM

(71) Applicant: LTS LOHMANN THERAPIE-SYSTEME AG, Andernach (DE)

(72) Inventors: Michael ORTH, Bad Honnef (DE); Tobias TELLKAMP-SCHEHR, Ratingen (DE); Florian HAMMES, Andernach (DE); Julia LODDER-GADACZEK, Selters Taunus (DE)

(73) Assignee: LTS LOHMANN THERAPIE-SYSTEME AG, Andernach (DE)

(21) Appl. No.: 17/631,629

(22) PCT Filed: Jul. 28, 2020
(86) PCT No.: PCT/EP2020/071312

§ 371 Date: Jan. 31, 2022

(30) Foreign Application Priority Data
Jul. 29, 2019 (DE) 10 2019 120 403.8

(51) Int. Cl. A61K 9/70; A61K 31/137; A61K 45/06

(52) U.S. Cl. CPC A61K 9/7061; A61K 31/137; A61K 45/06

(57) ABSTRACT

The invention relates to a process for preparing an active-principle-containing adhesive formulation for use in a transdermal therapeutic system, comprising the following steps: a) providing an adhesive formulation containing a carboxylic acid ester-based solvent and a self-adhesive polymer matrix containing monomers of the compounds on which the polymer matrix is based, b) removing the carboxylic acid ester-based solvent and the monomers from the adhesive formulation, c) re-dissolving the compound obtained in step b) in an organic solvent that does not contain any ester groups, and adding a pharmacologically active principle having at least one hydroxyl group, carboxyl group, amino group and/or ester group so that the active-principle-containing adhesive formulation is obtained. The invention also relates to a transdermal therapeutic system containing the active-principle-containing adhesive formulation. The invention further relates to the use of said transdermal therapeutic system and to a kit containing the transdermal therapeutic system.

The present invention relates to a process for producing an active-ingredient-containing adhesive formulation for use in a transdermal therapeutic system and also to a transdermal therapeutic system comprising this active-ingredient-containing adhesive formulation. The invention also relates to the use of a transdermal therapeutic system and to a kit comprising this transdermal therapeutic system.

Transdermal therapeutic systems (hereinafter “TTS” for short) are sheetlike pharmaceutical products with a layered construction in which at least one pharmacologically active ingredient is embedded with or without excipients (e.g., penetration enhancers) in an optionally pressure-sensitive adhesive polymer matrix. This polymer matrix is generally produced by coating a carrier film with the polymer composition comprising the active ingredient and then providing this system with a liner film which remains on the skin during application of the transdermal therapeutic system as well. The carrier film serves as a protective layer for the polymer matrix during the storage period and optionally as an aid to application for the subsequent use of the transdermal therapeutic system.

Transdermal therapeutic systems enable continuous supply of active ingredient over the entire application period. In terms of their concentration-time profiles, therefore, they are comparable with continuous drip infusions. Transdermal therapeutic systems are used typically for medical indications requiring the administration of an active ingredient over an unlimited or at least prolonged period, such as, for example, in the case of chronic diseases or in the case of hormone replacement therapy. Numerous transdermal therapeutic systems with different active ingredients or active-ingredient combinations are presently on the pharmaceutical market.

A range of commercially available transdermal therapeutic systems are constructed as what are called matrix systems. In these systems, the polymer matrix, which is or is not furnished so as to be pressure-sensitive adhesive, comprises the active ingredient in dissolved or suspended form. Acrylate-based pressure-sensitive adhesives highly regarded for use on the skin are available, for example, under the brand name DURO-TAK® and GELVA® (both from Henkel AG & Co. KGaA). These commercially available pressure-sensitive adhesives (PSAs) are sold as solutions in solvent and as part of conventional production operations are first of all mixed with at least one active ingredient and possibly excipients and adjuvants and applied to a carrier, and then the solvent is removed, to give a pressure-sensitive adhesive polymer matrix on the carrier. In the majority of cases, commercially available PSAs comprise a carboxylic ester-based solvent—in particular, the acrylate-based PSAs available under the brand name DURO-TAK® and GELVA®. Furthermore, commercially available PSAs also comprise monomers not reacted during the synthesis of the polymer matrix. As one such unreacted monomer, the PSAs available under the brand name DURO-TAK® and GELVA® contain first and foremost vinyl acetate.

It has been found, however, that when commercially available PSAs of these kinds are used for producing transdermal therapeutic systems, a portion of the active ingredient, added to the PSA in order to produce the transdermal therapeutic system, is no longer detectable in the polymer matrix just a short time after the end of the production operation. Moreover, just a short time after the production operation, reaction products have been detected in the polymer matrix that have very likely come about as a result of reactions and/or interactions between the pharmacologically active ingredient and the polymer matrix and/or solvents contained therein and/or monomers from the polymer matrix of the PSA.

Given that active ingredients used in transdermal therapeutic systems are in some cases very expensive, the reduced availability of the pharmacologically active ingredient in transdermal therapeutic systems is uneconomic in the case of production processes employing commercially available PSAs. Moreover, with transdermal therapeutic systems which are intended to remain on the skin over a prolonged period, it is necessary for these systems to comprise sufficient active ingredient to ensure that the therapeutically required amount of active ingredient is delivered over the entire duration of use, including toward the end of the period of use. Furthermore, reactions and/or interactions between the pharmacologically active ingredient and the polymer matrix and/or solvents contained therein reduce the storage stability of the transdermal therapeutic system, meaning that there is less active ingredient in the transdermal therapeutic system after storage. In addition, the reaction products resulting from the reactions and/or interactions between the pharmacologically active ingredient and the polymer matrix and/or solvents contained therein and/or monomers from the polymer matrix may possibly give rise to irritation on the skin. The prior art has disclosed a variety of approaches to increasing the amount of available active ingredient:

DE 10 141 652 A1 discloses a transdermal therapeutic system with a polymer matrix based on polyacrylate PSAs, and addresses the problem of the availability of the pharmacologically active ingredient. The publication teaches that a small fraction of monomers containing hydroxyl and/or carboxyl groups in the polymer matrix increases the availability of the active ingredient.

EP 2 158 905 discloses a transdermal therapeutic system which is intended to ensure sufficient delivery of the pharmacologically active ingredient to the skin. The PSA contained therein is based on polyacrylate. This publication, however, relates only to fentanyl as an active ingredient.

The solutions stated above are not always satisfactory. In studies leading to the present invention, for instance, it emerged that there are in some cases unwanted interactions between the PSA and certain pharmacologically active ingredients, meaning that the amount of active ingredient delivered in use is observed to be less than that actually expected. In some cases, moreover, there are signs of chemical degradation of the active ingredient in the presence of certain compounds in the adhesive formulation.

It is an object of the present invention to provide a process for obtaining an active-ingredient-containing adhesive formulation for use in a transdermal therapeutic system, wherein a greater part of the amount of active ingredient introduced during the production operation is available than in the case of conventional production processes. It is the intention, moreover, with the aid of the process, to obtain an active-ingredient-containing adhesive formulation which contains fewer reaction products resulting from interactions between the pharmacologically active ingredient and the polymer matrix and/or solvents contained therein. Furthermore, the process shall increase the storage stability of the active-ingredient-containing adhesive formulation. The transdermal therapeutic systems are preferably to (be able to) remain on the skin over a prolonged period, but at least 7 days.

This object has been achieved by means of a process for producing an active-ingredient-containing adhesive formulation for use in a transdermal therapeutic system, comprising the following steps:
a) providing an adhesive formulation comprising a carboxylic ester-based solvent and a pressure-sensitive adhesive polymer matrix which comprises monomers of the compounds on which the polymer matrix is based,
b) removing the carboxylic ester-based solvent and the monomers from the adhesive formulation,
c) redissolving the composition obtained in step b) in an organic solvent which contains no ester groups, and adding a pharmacologically active ingredient which has at least one hydroxyl group, carboxyl group, amino group and/or ester group, to give the active-ingredient-containing adhesive formulation.

The invention is based on the finding that by replacing the carboxylic ester-based solvent typically used in commercially customary adhesive systems for medical applications by a solvent without ester groups, it is possible to reduce or even prevent interactions with the pharmacologically active ingredient, so that the amount of active ingredient actually available for uptake via the skin is greater and, in particular, can be calculated more effectively. The explanation for these interactions may possibly—without being tied to this theory—be (esterification) reactions with hydroxyl, carboxyl or amino groups, or transesterification reactions with ester groups of the pharmacologically active ingredient.

According to one preferred embodiment of the process of the invention, the carboxylic ester-based solvent is selected from methyl acetate, ethyl acetate or butyl acetate, with the carboxylic ester-based solvent being preferably ethyl acetate.

The residual contents of the carboxylic ester-based solvent and/or of the monomers after this step b) may independently of one another be <1.0 wt %, based on the composition obtained in step b), preferably <0.5 wt %. The residual content of the carboxylic ester-based solvent is preferably in the range from 0.01 to 1.0 wt %, more preferably from 0.1 to 1.0 wt %.

In the process of the invention the organic solvent is preferably tetrahydrofuran, methanol, isopropanol, n-heptane, hexane, toluene, methyl ethyl ketone or ethanol. These solvents are typically inert toward the pharmacologically active ingredients and are suitable simultaneously for producing the adhesive coatings, since they are able to dissolve the customary adhesive matrices and, moreover, are sufficiently volatile for the subsequent removal.

As the polymer matrix it is possible in principle to select all types known to the skilled person for the production of TTS. These types are preferably selected from acrylates, pressure-sensitive silicone adhesives, polyisobutylene, SIS copolymers, silicone-acrylate hybrid systems, or mixtures thereof, with the polymer matrix preferably being a copolymer of 2-ethylhexyl acrylate, glycidyl methacrylate, 2-hydroxyethyl acrylate and/or vinyl acetate. With particular preference the polymer matrix has side chains with hydroxyl and/or carboxylic groups.

The pharmacologically active ingredient is preferably selected from fingolimod, ozanimod, teriflunomide, baclofen or cladribine, with the pharmacologically active ingredient preferably being fingolimod. These active ingredients typically exhibit increased interactions with the commercially customary ethyl acetate-based adhesives and are therefore preferred in the process of the invention.

Within the process of the invention provision may further be made to add in step c) at least one additive, the additive used preferably comprising a solubilizer for the active ingredient in the polymer matrix, a penetration enhancer, an antioxidant, a plasticizer, a bond strength enhancer, crosslinkers or mixtures of these. Employed as solubilizer for the active ingredient, preferentially, is a polymer different from the polymer matrix, preferably polyvinylpyrrolidone.

A further subject of the present invention relates to a transdermal therapeutic system (TTS) comprising
a carrier coated over very largely its full area with an active-ingredient-containing adhesive formulation produced by the process of the invention,
   optionally a protective film which, after the organic solvent has been removed from the layer of the active-ingredient-containing adhesive formulation, is applied over very largely the full area of this layer.

The invention further relates to a process for producing a transdermal therapeutic system, comprising the steps of
a) applying an active-ingredient-containing adhesive formulation produced by the process of the invention to a carrier, to give a layer covering very largely the full area of the carrier,
b) removing the organic solvent,
c) optionally applying a protective film on the side of the layer applied in step a) that is remote from the carrier, with the protective film covering very largely the full area of the layer.

The invention relates, moreover, to the use of a transdermal therapeutic system of the invention for the treatment of hypogonadism, for hormone replacement therapy, for the treatment of Alzheimer's, of Parkinson's, of Multiple Sclerosis, of bipolar disorders, of muscle strains, of pain, of hypertension or for contraception, with the use of a transdermal therapeutic system comprising fingolimod as pharmacologically active ingredient for the treatment of Multiple Sclerosis or of diseases treated by immunosuppression being preferred.

A further subject of the present invention is a transdermal therapeutic system of the invention for use in a process for the treatment of hypogonadism, for hormone replacement therapy, for the treatment of Alzheimer's, of Parkinson's, of Multiple Sclerosis, of bipolar disorders, of muscle strains, of pain, of hypertension or for contraception, with a transdermal therapeutic system comprising fingolimod as pharmacologically active ingredient for use in a process for the treatment of Multiple Sclerosis or of diseases treated by immunosuppression being preferred.

The invention also relates to a kit comprising a transdermal therapeutic system of the invention in an outer packaging and optionally use instructions for the use of the transdermal therapeutic system.

The invention relates in particular to the following embodiments:

According to a first embodiment, the invention relates to a process for producing an active-ingredient-containing adhesive formulation for use in a transdermal therapeutic system, comprising the following steps:
a) providing an adhesive formulation comprising a carboxylic ester-based solvent and a pressure-sensitive adhesive polymer matrix which comprises monomers of the compounds on which the polymer matrix is based,
b) removing the carboxylic ester-based solvent and the monomers from the adhesive formulation,
c) redissolving the composition obtained in step b) in an organic solvent which contains no ester groups, and adding a pharmacologically active ingredient which has at least one hydroxyl group, carboxyl group, amino group and/or ester group, to give the active-ingredient-containing adhesive formulation.

According to a second embodiment, the invention relates to a process according to embodiment 1, characterized in that the carboxylic ester-based solvent is selected from methyl acetate, ethyl acetate or butyl acetate, with the carboxylic ester-based solvent being preferably ethyl acetate.

According to a third embodiment, the invention relates to a process according to embodiment 1 or 2, characterized in that the residual contents of the carboxylic ester-based solvent and/or of the monomers after this step b) independently of one another are each <1.0 wt %, based on the composition obtained in step b), preferably <0.5 wt %.

According to a fourth embodiment, the invention relates to a process according to any of the preceding embodiments, characterized in that the organic solvent is tetrahydrofuran, methanol, isopropanol, n-heptane, hexane, toluene, methyl ethyl ketone or ethanol.

According to a fifth embodiment, the invention relates to a process according to any of the preceding embodiments, characterized in that the polymer matrix is selected from acrylates, pressure-sensitive silicone adhesives, polyisobutylene, SIS copolymers, silicone-acrylate hybrid systems, or mixtures thereof, with the polymer matrix preferably being a copolymer of 2-ethylhexyl acrylate, glycidyl methacrylate, 2-hydroxyethyl acrylate and/or vinyl acetate.

According to a sixth embodiment, the invention relates to a process according to embodiment 5, characterized in that the polymer matrix has side chains with hydroxyl and/or carboxyl groups.

According to a seventh embodiment, the invention relates to a process according to any of the preceding embodiments, characterized in that the pharmacologically active ingredient is selected from fingolimod, ozanimod, teriflunomide, baclofen or cladribine, with the pharmacologically active ingredient preferably being fingolimod.

According to an eighth embodiment, the invention relates to a process according to any of the preceding embodiments, characterized in that at least one additive is added in step c), with the additive used preferably comprising a solubilizer for the active ingredient in the polymer matrix, a penetration enhancer, an antioxidant, a plasticizer, a bond strength enhancer, crosslinkers or mixtures of these.

According to a ninth embodiment, the invention relates to a process according to embodiment 8, characterized in that the solubilizer for the active ingredient is a polymer different from the polymer matrix, preferably polyvinylpyrrolidone.

According to a tenth embodiment, the invention relates to a transdermal therapeutic system comprising
a carrier coated over very largely its full area with an active-ingredient-containing adhesive formulation produced by a process according to any of embodiments 1 to 9,
optionally a protective film which, after the organic solvent has been removed from the layer of the active-ingredient-containing adhesive formulation, is applied over very largely the full area of this layer.

According to an eleventh embodiment, the invention relates to a process for producing a transdermal therapeutic system, comprising the steps of
a) applying an active-ingredient-containing adhesive formulation produced by a process according to any of embodiments 1 to 9 to a carrier, to give a layer covering very largely the full area of the carrier,
b) removing the organic solvent,
c) optionally applying a protective film on the side of the layer applied in step a) that is remote from the carrier, with the protective film covering very largely the full area of the layer.

According to a twelfth embodiment, the invention relates to a use of a transdermal therapeutic system according to embodiment 10 for the treatment of hypogonadism, for hormone replacement therapy, for the treatment of Alzheimer's, of Parkinson's, of Multiple Sclerosis, of bipolar disorders, of muscle strains, of pain, of hypertension or for contraception, with the use of a transdermal therapeutic system comprising fingolimod as pharmacologically active ingredient for the treatment of Multiple Sclerosis or of diseases treated by immunosuppression being preferred.

According to a thirteenth embodiment, the invention relates to a transdermal therapeutic system according to embodiment 10 for use in a process for the treatment of hypogonadism, for hormone replacement therapy, for the treatment of Alzheimer's, of Parkinson's, of Multiple Sclerosis, of bipolar disorders, of muscle strains, of pain, of hypertension or for contraception, with a transdermal therapeutic system comprising fingolimod as pharmacologically active ingredient for use in a process for the treatment of Multiple Sclerosis or of diseases treated by immunosuppression being preferred.

According to a fourteenth embodiment, the invention relates to a kit comprising a transdermal therapeutic system according to embodiment 10 in an outer packaging and optionally use instructions for the use of the transdermal therapeutic system according to embodiment 12 or 13.

### EXAMPLES

The present invention is elucidated further by means of the examples below, but without being limited to them.

Various adhesive formulations were produced, using different commercially available pressure-sensitive adhesives (PSAs).

TABLE 1
Commercially available PSAs used

| | | Polymer | | Contains | |
|---|---|---|---|---|---|
| | Trade- | containing | Functional | vinyl | Solvent in |
| PSA | name | as monomers | groups | acetate | the PSA |
| 1 | Durotak | 2-ethylhexyl acrylate, | OH groups | Yes | Ethyl |
| | 387-2287 | vinyl acrylate | | | acetate |
| | | 2-hydroxyethyl acrylate | | | |
| | | glycidyl methacrylate | | | |
| 2 | Durotak | 2-ethylhexyl acrylate, | OH groups | Yes | Ethyl |
| | 87-4287 | vinyl acrylate | | | acetate |
| | | 2-hydroxyethyl acrylate | | | |

PSAs 1 and 2 are suitable for TTS which are worn on the skin for at least 7 days.

Production of the Adhesive Formulations of the Invention

The commercially available PSAs stated above were coated out onto siliconized films and the resulting laminates were dried at room temperature for 15 minutes and thereafter at 60° C. for 10 minutes. After drying, the dried PSAs were removed from the films and redissolved in an organic solvent which contains no ester groups, and 3 wt %, based on the total mass of the dried PSA, of pharmacologically active ingredient, and also, optionally, 10 wt % of polyvinylpyrrolidone, were added. After the resulting compositions had been homogenized, they were coated out onto siliconized films to give a laminate. The laminates were dried at room temperature for 15 minutes and thereafter at 60° C. for 10 minutes, to give an adhesive formulation. The respective composition of the TTS of the invention is reported in table 2.

TABLE 2
Composition of the adhesive formulations of the invention

| | | | | Solvent for the |
|---|---|---|---|---|
| Inventive | | Pharm. act. | Polyvinyl- | redissolution |
| ex. | PSA | Ingredient | pyrrolidone | after drying |
| 1 | 1 | Fingolimod | No | Ethanol |
| 2 | 1 | Fingolimod | Yes | Tetrahydrofuran |
| 3 | 2 | Fingolimod | Yes | Tetrahydrofuran |

TABLE 3
Visual assessment of the compositions of the invention after
addition of the active ingredient and second drying

| | Appearance of the | | |
|---|---|---|---|
| Inventive | composition before | | Appearance of |
| ex. | coating out | Viscosity | the laminate |
| 1 | Homogeneous, cloudy, | Highly viscous | Homogeneous, cloudy |
| | milky | | |
| 2 | Homogenous, cloudy | Moderately | Crystals perceptible, |
| | | viscous | cloudy |
| 3 | Homogenous, cloudy | Highly viscous | Crystals perceptible, |
| | | | cloudy |

Production of the Reference Examples

The commercially available PSAs were admixed with in each case 3 wt %, based on the total mass of the PSA, of pharmacologically active ingredient, and also, in some cases, 10 wt % of polyvinylpyrrolidone. Following homogenization of the resultant compositions, they were coated out onto siliconized films to give a laminate. The laminates were dried at room temperature for 15 minutes and thereafter at 60° C. for 10 minutes, to give an adhesive formulation.

TABLE 4
Composition of reference examples

| Reference | | Pharm. act. | Polyvinyl- |
|---|---|---|---|
| ex. | PSA | ingredient | pyrrolidone |
| 2a | 1 | Fingolimod | No |
| 3a | 1 | Fingolimod | Yes |
| 4a | 2 | Fingolimod | Yes |

Investigation of the Adhesive Formulations Obtained

The fraction of pharmacologically active ingredient in the laminates obtained was extracted using an acidified methanol (1 vol % of conc. nitric acid in methanol). The extracts were diluted with a phosphate buffer, pH 2.5, and then analyzed by means of HPLC gradient elution on a C8 phase with diode array detection at 220 nm. The eluents used were the following mixtures:

Eluent A: 0.01-M KH₂PO₄:acetonitrile (80:20, v/v), 0.1% triethylamine, pH 2.5

Eluent B: 0.01-M KH₂PO₄:acetonitrile (20:80, v/v), 0.05% triethylamine, pH 2.5

Gradient profile: t[min]/B[%]: 0/35, 6/37, 8/40, 12/55, 18/100, 22/100, 24/35, 28/35

Quantification took place on a calibration plot drawn up using fingolimod reference material (external standard).

Table 5 below reports the fraction of fingolimod and any degradation products or byproducts of the fingolimod found in the adhesive formulations. Byproducts reported were degradation products and byproducts of the active ingredient whose UV spectrum is similar to that of fingolimod. To identify the byproducts, LC/MS and LCMS/MS were carried out on 1:1 mixtures of fingolimod with ethyl acetate and, respectively, with vinyl acetate in solution in methanol, and structural resolutions were conducted, resulting in the following byproducts:
acetamide of fingolimod, or acetyl-fingolimod (both have the same molar mass of M=350, it therefore not being known which of the two reaction products is present),
vinyl-fingolimod, M=334, and
reaction product of fingolimod with 2 vinyl groups, M=360.

The percentages >100% derive firstly from a measurement inaccuracy of ±2% and secondly from the fact that inaccuracies of 1-2% in relation to the amount of fingolimod may occur when the adhesive formulations are produced on the laboratory scale.

TABLE 5
Fraction of fingolimod and any degradation products or byproducts
of fingolimod in the adhesive formulations obtained

| | Fraction of | Byproducts of |
|---|---|---|
| | fingolimod | fingolimod |
| | [%] | [%] |
| Inventive 1 | 102 | 0.3 |
| Reference 1a | 98 | 2.0 |
| Inventive 2 | 99 | 0.7 |
| Reference 2a | 98 | 2.0 |
| Inventive 3 | 103 | 0.4 |
| Reference 3a | 97 | 1.2 |

[%] based on amounts of fingolimod added

The adhesive formulations of the invention had a higher fraction of fingolimod than those of the reference examples. Furthermore, the fraction of byproducts resulting from reactions of fingolimod with solvents or residual monomers from the commercially available PSAs is significantly reduced for the adhesive formulations of the invention. From the results, therefore, it is apparent that the removal of the residual monomers, in this case vinyl acetate, and of the solvent, in this case ethyl acetate, from the commercially available adhesives means that these compounds are no longer available as reaction partners for the active ingredient, presently fingolimod, and therefore that a higher recovery rate of the active ingredient in the adhesive formulations, and also a smaller fraction of byproducts, are detectable. The results thus show that in the case of the adhesive formulations of the invention, the availability of the ingredient is higher than with commercially available, known PSAs.

Aging Behavior of PSAs Containing Ethyl Acetate and Vinyl Acetate

In order to show that the residual monomers and solvents contained in the commercially available PSAs react with the active ingredient fingolimod and contribute to a reduced availability of the active ingredient after storage, LC/MS and LCMS/MS were carried out on 1:1 mixtures of fingolimod with ethyl acetate, or with vinyl acetate, in solution in methanol, and the formulation of reaction products of fingolimod with ethyl acetate and vinyl acetate, and also of the decomposition products of fingolimod, was investigated. Storage took place under laboratory conditions at room temperature.

TABLE 6
Reaction products of the active ingredient with
residual monomers or solvent after storage

| | | | Number of | |
|---|---|---|---|---|
| | | | byproducts with a | |
| Fingolimod | Age of | | fraction > 0.2% | Sum total of |
| with | sample | | in the solution | all byproducts |
| Vinyl acetate | 2 | days | 8 | around 57% |
| Vinyl acetate | 1 | month | 16 | around 44% |
| Ethyl acetate | 2 | days | 3 | around 15% |

Just 2 days after production, a multiplicity of decomposition products and reaction products of fingolimod with the compounds contained as residual monomers or solvents in commercially available PSAs were perceptible. In particular the concentration of a number of reaction products, at >0.2%, was very high.

The results of investigation on the fingolimod/vinyl acetate solution showed that further byproducts form after one month, but with no rise in the overall concentration of byproducts. When interpreting the test results after 1 month of storage, however, it should be borne in mind that probably not all of the compounds could be detected, since their structure is possibly not known to the HPLC-FLD detector.

1. A process for producing an active-ingredient-containing adhesive formulation for use in a transdermal therapeutic system, comprising the following steps:
a) providing an adhesive formulation comprising a carboxylic ester-based solvent and a pressure-sensitive adhesive polymer matrix which comprises monomers of the compounds on which the polymer matrix is based,
b) removing the carboxylic ester-based solvent and the monomers from the adhesive formulation to obtain a composition, and
c) redissolving the composition obtained in step b) in an organic solvent which contains no ester groups, and adding a pharmacologically active ingredient which has at least one hydroxyl group, carboxyl group, amino group and/or ester group, to give the active-ingredient-containing adhesive formulation.

2. The process as claimed in claim 1, wherein the carboxylic ester-based solvent is selected from methyl acetate, ethyl acetate or butyl acetate.

3. The process as claimed in claim 1, wherein residual contents of the carboxylic ester-based solvent and/or of the monomers after this-step b) independently of one another are each <1.0 wt %, based on the composition obtained in step b), preferably <0.5 wt %.

4. The process as claimed in claim 1, wherein the organic solvent is tetrahydrofuran, methanol, isopropanol, n-heptane, hexane, toluene, methyl ethyl ketone, or ethanol.

5. The process as claimed in claim 1, wherein the polymer matrix is selected from acrylates, pressure-sensitive silicone adhesives, polyisobutylene, SIS copolymers, silicone-acrylate hybrid systems, or mixtures thereof.

6. The process as claimed in claim 5, wherein the polymer matrix has side chains with hydroxyl and/or carboxyl groups.

7. The process as claimed in claim 1, wherein the pharmacologically active ingredient is selected from fingolimod, ozanimod, teriflunomide, baclofen, or cladribine.

8. The process as claimed in claim 1, wherein at least one additive is added in step c).

9. The process as claimed in claim 8, which further comprises adding a solubilizer for the active ingredient, wherein the solubilizer for the active ingredient is a polymer different from the polymer matrix.

10. A transdermal therapeutic system comprising
a carrier coated over very largely its full area with an active-ingredient-containing adhesive formulation produced by a process as claimed in claim 1, and
optionally a protective film which, after the organic solvent has been removed from the layer of the active-ingredient-containing adhesive formulation, is applied over very largely the full area of this layer.

11. A process for producing a transdermal therapeutic system, comprising the steps of
a) applying an active-ingredient-containing adhesive formulation produced by a process as claimed in claim 1 to a carrier, to give a layer covering very largely the full area of the carrier,
b) removing the organic solvent, and
c) optionally applying a protective film on a side of the layer applied in step a) that is remote from the carrier, with the protective film covering very largely the full area of the layer.

12. A method for the purpose of treatment of hypogonadism, for hormone replacement therapy, for the treatment of Alzheimer's, of Parkinson's, of Multiple Sclerosis, of bipolar disorders, of muscle strains, of pain, of hypertension, or for contraception, in a patient in need thereof, said method comprising (A) providing a transdermal therapeutic system as claimed in claim 10, wherein the pharmacologically active ingredient is effective for said purpose, and (B) applying said transdermal therapeutic system to the skin of said patient.

13. The method as claimed in claim 12, wherein fingolimod is the pharmacologically active ingredient and the transdermal therapeutic system is applied for the purpose of treatment of Multiple Sclerosis or of diseases treated by immunosuppression.

14. A kit comprising a transdermal therapeutic system as claimed in claim 10 in an outer packaging and optionally usage instructions for the use of the transdermal therapeutic system.

15. The process as claimed in claim 2, wherein the carboxylic ester-based solvent is ethyl acetate.

16. The process as claimed in claim 3, wherein residual contents of the carboxylic ester-based solvent and/or of the monomers after this step b) independently of one another are each <0.5 wt %, based on the composition obtained in step b).

17. The process as claimed in claim 5, wherein the polymer matrix comprises a copolymer of 2-ethylhexyl acrylate, glycidyl methacrylate, 2-hydroxyethyl acrylate and/or vinyl acetate.

18. The process as claimed in claim 7, wherein the pharmacologically active ingredient is fingolimod.

19. The process as claimed in claim 8, wherein the additive comprises a solubilizer for the active ingredient in the polymer matrix, a penetration enhancer, an antioxidant, a plasticizer, a bond strength enhancer, crosslinkers, or a mixture of these.

20. The process as claimed in claim 9, wherein the polymer different from the polymer matrix is polyvinylpyrrolidone.
